(19) United States
(12) Patent Application Publication
(10) Pub. No.: US 2025/0359785 A1
(43) Pub. Date: Nov. 27, 2025

(54) ESTIMATING A CONCENTRATION OF A RESPIRATORY GAS IN THE BLOOD OF A PATIENT

(71) Applicant: Loewenstein Medical Technology S.A., Luxembourg (LU)

(72) Inventors: Peter KREMEIER, Karlsruhe (DE); Gerardo TUSMAN, Mar del Plata (AR)

(21) Appl. No.: 19/215,710

(22) Filed: May 22, 2025

(30) Foreign Application Priority Data
May 27, 2024 (DE) 10 2024 114 793.8

(51) Int. Cl. A61B 5/145; A61B 5/00; A61B 5/083; A61B 5/091

(52) U.S. Cl. CPC A61B 5/14542; A61B 5/083; A61B 5/091; A61B 5/7267

(57) ABSTRACT

A method for estimating a concentration of a respiratory gas in the blood of a patient comprises: receiving measurement data, which indicate a volume-dependent course of a concentration of the respiratory gas in a respiratory airflow exhaled by the patient depending on a respiratory air volume exhaled by the patient; generating input data from the measurement data, the input data comprising a matrix of values for various parameters with respect to the volume-dependent course; inputting the input data into a machine learning module which was trained to convert the input data into output data, which indicate a concentration of the respiratory gas in the blood of the patient; outputting the output data by way of the machine learning module.

### CROSS-REFERENCE TO RELATED APPLICATIONS

The present application claims priority under 35 U.S.C. § 119 of German Patent Application No. 10 2024 114 793.8, filed May 27, 2024, the entire disclosure of which is expressly incorporated by reference herein.

### BACKGROUND OF THE INVENTION

### 1. Field of the Invention

The invention relates to a method for estimating a concentration of a respiratory gas in the blood of a patient. Moreover, the invention relates to a method for training a machine learning module for use in such a method. Furthermore, the invention relates to a data processing device, a computer program, and a computer-readable medium for carrying out at least one of these methods and a medical device.

### 2. Discussion of Background Information

In the machine ventilation of anesthetized and critically ill patients, the proper exchange of biological gases has to be continuously maintained. The analysis of biological gases in arterial blood samples represents the gold standard for assessing the gas exchange in the clinical sector. In this case, the partial pressures of carbon dioxide and oxygen in the blood are compared to the proportion of the inhaled oxygen and the alveolar ventilation to establish whether or not respiratory failure exists. However, such a blood gas analysis is invasive and time-consuming.

In addition, it is possible to measure the alveolar carbon dioxide partial pressure (p_{A}CO₂) noninvasively by means of capnography. However, such a measurement has not been able to replace a conventional blood gas analysis—in particular in anesthetized and critically ill patients—up to this point.

In view of the foregoing, it would be advantageous to have available a method which enables a concentration of a respiratory gas in the blood of a patient to be determined noninvasively with sufficient accuracy. It further would be advantageous to have available a method for training a corresponding machine learning module, a corresponding data processing device, a corresponding computer program, a corresponding computer-readable medium, and a corresponding medical device.

### SUMMARY OF THE INVENTION

In a first aspect, the present invention provides a computer-implemented method for estimating a concentration of a respiratory gas in the blood of a patient. The method comprises: receiving measurement data, which indicate a volume-dependent course of a concentration of the respiratory gas in a respiratory airflow exhaled by the patient depending on a respiratory air volume exhaled by the patient; generating input data from the measurement data, wherein the input data comprise a matrix of values for various parameters with respect to the volume-dependent course; inputting the input data into a machine learning model, which was trained to convert the input data into output data, wherein the output data indicate a concentration of the respiratory gas in the blood of the patient; outputting the output data by way of the machine learning module.

In a second aspect, the invention further provides a computer-implemented method for training a machine learning module for a medical device. The method comprises: receiving multiple measurement data sets, which each comprise measurement data that indicate a volume-dependent course of a concentration of a respiratory gas in a respiratory airflow exhaled by a patient depending on a respiratory air volume exhaled by the patient, wherein the measurement data of various measurement data sets are each at least partially associated with different patients; generating multiple training data sets from the measurement data sets, wherein each training data set is associated with one of the patients and comprises a matrix of values for various parameters with respect to the volume-dependent course; inputting each training data set as input data into the machine learning module, which is configured to convert the input data into output data, wherein the output data indicate a concentration of the respiratory gas in the blood of the respective patient; outputting the output data by way of the machine learning module; determining a deviation of the output data from target data which are assigned to the respective training data set (underlying the output data); adapting weights of the machine learning module in an optimization method in order to reduce the deviation.

The method may be carried out automatically by a processor, for example by a processor of a medical device. The machine learning module used in the method according to the first aspect of the invention can have been trained using the method according to the second aspect of the invention. The method according to the first aspect of the invention can additionally comprise the steps of the method according to the second aspect of the invention.

The approach presented here is based on the finding that to estimate a concentration of a respiratory gas in the blood of a patient, in particular in their arterial blood, the curve of a volumetric capnogram or oxigram can be analyzed with the aid of a machine learning algorithm. This curve changes depending on variations in the lung ventilation and perfusion of the patient. Such variations can result in greater inaccuracies in the case of estimation using conventional methods.

In contrast, the methods described above and below also permit a very accurate estimation of the concentration of the respiratory gas in the blood of the patient in various patients and/or with strong variations of the lung function. This has the advantage that an invasive blood gas analysis has to be carried out less frequently or can even be dispensed with.

In comparison to an embodiment in which the concentration of the respiratory gas in the blood of the patient is estimated directly on the basis of the (raw) measurement data or in which the (raw) measurement data are used as the input data, the methods described above and below moreover have the advantage that significantly less computing power is necessary and a sufficiently good estimation is enabled even with measurement data of lower quality. Moreover, the risk of misinterpretations is reduced, because the input data are predefined in contrast to (raw) measurement data.

It is particularly advantageous if multiple parameters are analyzed with respect to the curve. Such a multivariate approach supplies more information for the estimation than if only one single parameter, for example the alveolar partial pressure of the relevant respiratory gas, is analyzed, and in combination with a correspondingly configured machine learning algorithm enables a significantly more accurate and robust estimation.

Several terms are explained in more detail hereinafter.

A “patient” can be understood as a ventilation patient, i.e., a human or animal subject who is ventilated or is supposed to be ventilated by means of a ventilator.

“Respiratory gas” can be understood, for example, as one of the following gases: carbon dioxide, oxygen, nitrogen, water vapor, anesthetic gas.

“Respiratory air” as in “respiratory airflow” or “respiratory air volume” can be understood as a respiratory gas mixture comprising the respiratory gas.

“Concentration” can be understood in general as a proportion or an amount, in particular a partial pressure.

The concentration of the respiratory gas in the blood of the patient can be, for example, an arterial and/or venous partial pressure of the respiratory gas.

The measurement data may have been at least partially generated using a corresponding sensor system, for example, a sensor system of a medical device. For example, the measurement data may have been generated noninvasively by capnography and/or oxigraphy. In other words, the measurement data may be, for example, data from a capnogram and/or an oxigram.

The measurement data underlying the training data sets may comprise real data (i.e. resulting from a real measurement) and/or simulated data. The simulated data—in contrast to the real data—may have been generated using a simulation environment, in which lung conditions of various patients are simulated by a computer.

“Input data” can be understood as data deviating from the measurement data and/or data especially adapted to the machine learning module, in contrast to the measurement data. In particular, the input data may be data compressed in relation to the measurement data. It is possible that the measurement data are input into a further machine learning module which was trained to convert the measurement data into the input data.

A “machine learning module” can be understood as a hardware and/or software module for converting input data into output data in an algorithm parameterized, i.e., trained by machine learning. Such an algorithm can be, for example, an artificial neural network, a decision tree, a random forest, a k-nearest neighbor algorithm, a support vector machine, a Bayes classifier, a k-means algorithm, a genetic algorithm, a kernel regression algorithm, a discriminant analysis algorithm, or combination of at least two of these examples.

Each training data set which is input into the machine learning module can be assigned a set of predefined target data. The target data may comprise, for example, a target value assigned to the respective training data set for the concentration of the respiratory gas in the blood of the respective patient. In particular, the target data may indicate a result of a measurement (for example, a blood gas analysis), which was carried out at the same time and/or shortly before and/or shortly after the time at which the measurement data underlying the respective training data set were generated. The target data may comprise real data (i.e. resulting from a real measurement) and/or simulated data. The simulated data—in contrast to the real data—may have been generated using a simulation environment, in which lung conditions of various patients are simulated by a computer, together with the respective training data set.

To determine the deviation of the output data from the target data, the output data and the target data may be input into a suitable loss function to calculate a score quantifying the deviation. The score may be calculated, for example, with the aid of the method of least squares.

An “optimization method” can be understood as an iterative method for minimizing the loss function, for example a gradient method, in particular a random gradient method, having back propagation.

A third aspect of the invention relates to a data processing device. The data processing device comprises a processor configured to carry out at least one of the methods described above and below.

A “data processing device” can be understood in general as a computer. The data processing device can comprise hardware and/or software components. The data processing device may be, for example, a controller, a PC, a server, a laptop, tablet, a smart phone, or a combination of at least two of these examples. Alternatively, a “data processing device” can be understood as at least one hardware and/or software component of at least one of these examples.

A “processor” can be understood, for example, as a CPU (central processing unit), a graphics processor, a TPU (tensor processing unit), or a combination of at least two of these examples.

In addition to the processor, the data processing device may comprise at least one of the following components: a memory, a bus system for data communication between the processor and the memory, a data communication interface for wireless and/or wired data communication with peripheral devices.

It is to be noted that features of the methods described above and below may also be features of the data processing device (and vice versa).

A fourth aspect of the invention relates to a medical device. The medical device comprises a sensor system for generating measurement data, which indicate a volume-dependent course of a concentration of a respiratory gas in a respiratory airflow exhaled by a patient depending on a respiratory air volume exhaled by the patient, as well as a data processing device as described above and below.

The medical device may be, for example, a ventilator for invasive and/or noninvasive ventilation of a patient and/or a monitoring monitor for monitoring vital parameters of a patient.

The sensor system may comprise one or more gas sensors. A “gas sensor” can be understood, for example, as a galvanic, paramagnetic, or optical sensor. Such a gas sensor may be arranged in a main flow and/or a secondary flow of the exhaled respiratory air and/or may be designed as a pressure and/or flow sensor.

Further aspects of the invention relate to a computer program and a computer-readable medium, on which the computer program is stored.

The computer program comprises commands which prompt a processor (for example, the processor of the data processing device described above and below), when the computer program is executed by the processor, to carry out at least one of the methods described above and below.

The computer-readable medium may be a volatile or nonvolatile data memory. For example, the computer-readable medium may be a hard drive, a USB (universal serial bus) storage device, a RAM (random-access memory), a ROM (read-only memory), an EPROM (erasable programmable read-only memory), an EEPROM (electrically erasable programmable read-only memory), a flash memory, or a combination of at least two of these examples. The computer-readable medium may also be a data communication network which enables the downloading of program code (for example, via the Internet), or a cloud.

It is to be noted that features of the methods described above and below may also be features of the computer program and/or the computer-readable medium (and vice versa).

Various embodiments of the invention are described hereinafter. These embodiments are not to be understood as a restriction of the scope of the invention.

According to one embodiment, the measurement data may indicate the volume-dependent course with respect to a single breath of the patient. In other words, each point of the volume-dependent course may be associated with a specific proportion of a total volume of respiratory air comprising the respiratory gas exhaled by the patient during a single breath.

Accordingly, a volume of zero may be associated with the beginning of the volume-dependent course and a volume equal to the total volume may be associated with the end of the volume-dependent course. Such a total volume may also be referred to as a breath volume or tidal volume. It is possible that measurement data were generated during the single breath and/or are generated again and/or received again upon each breath.

In other words, the measurement data may comprise an array of concentration values for the concentration of the respiratory gas in the respiratory airflow and an array of volume values for the respiratory air volume. Each volume value may be a value from a value range bounded by a lower limiting value and an upper limiting value, wherein the lower limiting value is zero and the upper limiting value indicates a total volume exhaled by the patient during a single breath and a different volume value is associated with each concentration value.

According to one embodiment, the measurement data may furthermore indicate a positive end-expiratory pressure, associated with the volume-dependent course, for the ventilation of the patient. In this case, the input data may comprise the positive end-expiratory pressure and/or may be generated in consideration of the positive end-expiratory pressure. This enables a more accurate estimation in comparison to an embodiment without consideration of the positive end-expiratory pressure.

According to one embodiment, a mathematical function, which approximately defines at least one section of the volume-dependent course, may be determined using the measurement data. At least one of the values in the matrix may be calculated here using the mathematical function. Suitable parameters may be determined in a predictable and transparent manner with the aid of the mathematical function. The mathematical function may be a single mathematical function or a combination of multiple individual mathematical functions.

According to one embodiment, the input data may comprise a matrix of values for 2 to 20, 10 to 20, or 10 to 15 different parameters with respect to the volume-dependent course. In this way, the consumption of computing resources can be significantly reduced in comparison to an embodiment having larger input matrices.

A “parameter” can be understood above and below as a ventilation parameter relevant for a ventilation of a patient. Each value in the matrix may be associated here with one of the different parameters. Accordingly, the matrix can comprise, for example, 2 to 20, 10 to 20, or 10 to 15 input values depending on the number of the different parameters. The matrix can be understood as a one-dimensional, two-dimensional, or three-dimensional vector. For example, the machine learning module may be configured to convert these input values into a single output value, which indicates the concentration of the respiratory gas in the blood of the patient.

According to one embodiment, the measurement data may have been generated in multiple successive time steps. In this case, the mathematical function may be determined using the measurement data from various time steps, for example by regression. A single time step may last, for example, as long as a single breath of the patient. The respective duration of the time steps may also be permanently predetermined, however, and/or can be, for example, in an order of magnitude of 1 ms, 10 ms, 100 ms, 1 second, or 10 seconds.

According to one embodiment, the mathematical function may be determined according to the Levenberg-Marquardt algorithm. The “Levenberg-Marquardt algorithm” can be understood as a special numerical optimization algorithm for solving nonlinear balancing problems with the aid of the method of least squares. The algorithm can be understood as a combination of the Gauss-Newton method with a regularization technique which forces decreasing function values. This enables a more accurate and computing-efficient approximation than implementation of the classic Fowler method, specifically even in the event of stronger variations of the volume-dependent course between successive breaths and/or between different patients.

According to one embodiment, the various parameters may comprise at least one of the following parameters: a total volume of the respiratory gas exhaled during a single breath by the patient; a total volume of respiratory air comprising the respiratory gas exhaled during a single breath by the patient (also called breath volume or tidal volume); a respiratory minute volume; an alveolar ventilation; an airway dead space, a mixed expiratory partial pressure of the respiratory gas, an end tidal partial pressure of the respiratory gas; a positive end-expiratory pressure for ventilating the patient.

The respiratory minute volume can be understood as the product of the breath volume and the respiratory frequency.

The alveolar ventilation (\dot{V}_{A}) can be understood as the product of the breathing rate and the difference between the breath volume and the (anatomical) dead space.

The “airway dead space” can be understood in general as a proportion of the breath volume which remains in the air-guiding airways during each breath and therefore does not reach the alveolar compartment.

The end-tidal partial pressure of the respiratory gas can be understood as the concentration of the respiratory gas in the respiratory airflow exhaled by the patient at the end of a single breath.

The mixed expiratory partial pressure of the respiratory gas can be understood as a specific fraction of the end-tidal partial pressure and/or, for example, may correspond to a mean concentration of the respiratory gas in the respiratory airflow exhaled by the patient in a single breath.

According to one embodiment, the volume-dependent course may be divided into at least three successive characteristic exhalation phases during a single breath of the patient. In this case, the various parameters may comprise at least one of the following parameters: a first respiratory gas volume as a volume of the respiratory gas exhaled by the patient in a first (for example, earliest) of the exhalation phases; a second respiratory gas volume as a volume of the respiratory gas exhaled by the patient in a second (for example, middle) of the exhalation phases; a third respiratory gas volume as a volume of the respiratory gas exhaled by the patient in a third (for example, last or next-to-last) of the exhalation phases; a (for example, average) slope of the volume-dependent course in at least one of the exhalation phases, in particular in a last (or next-to-last) of the exhalation phases.

The middle exhalation phase can be understood as an exhalation phase lying between the earliest and the last (or next-to-last) exhalation phase with respect to time. It is possible that the first exhalation phase merges directly into the second exhalation phase and/or the second exhalation phase merges directly into the third exhalation phase.

A “slope” can be understood as a (for example, average) rate of change of the concentration of the respiratory gas in the respiratory gas flow with respect to the respective exhalation phase(s). The slope may additionally be normalized in a suitable manner to obtain a normalized slope, which can then be used as one of the parameters.

The exhalation phases may have been determined, for example, with the aid of the above-mentioned mathematical function and/or according to the Fowler method. The exhalation phases may be the typical (three or four) phases of a volumetric capnogram or oxigram.

The exhalation phases may differ from one another significantly in their length and/or in the (for example, average) slope of the volume-dependent course.

For example, in the case of a capnogram, the first exhalation phase may extend from the beginning of the exhalation to a first point at which the rate of change of the second derivative of the volume-dependent course reaches its maximum or the third derivative of the volume-dependent course reaches its left-side maximum. The second exhalation phase may extend from the first point to a second point, at which the third derivative of the volume-dependent course reaches its right-side maximum. The third exhalation phase may extend from the second point to the end of the exhalation. The term “volume-dependent course” can also be understood here as an approximation, for example, in the form of the above-mentioned mathematical function.

The first exhalation phase may be the earliest phase of the exhalation (10% to 12% of the total breath), in which hardly any or no carbon dioxide is contained in the respiratory air. The second exhalation phase may be a phase of the greatest (average) increase of the carbon dioxide concentration in the respiratory airflow (15% to 18% of the total breath). The third exhalation phase may be a phase in which the carbon dioxide concentration in the respiratory airflow—in contrast to the preceding exhalation phases—is predominantly determined by the gases coming out of the alveoli (70% to 75% of the total breath).

According to one embodiment, at least one normalized respiratory gas volume may be determined by dividing one of the three respiratory gas volumes by a total volume (of the respiratory gas or respiratory air comprising the respiratory gas) exhaled by the patient during the single breath. In this case, the parameters may comprise the at least one normalized respiratory gas volume additionally or alternatively to the respective (not normalized) first, second, or third respiratory gas volume.

According to one embodiment, the machine learning module may comprise an artificial neural network. Accordingly, the weights of the machine learning module may be weights of the artificial neural network. The artificial neural network may comprise an input layer for inputting the input data, an output layer for outputting the output data, and at least one trainable intermediate layer between the input layer and the output layer. The artificial neural network may comprise, for example, at least one of the following network types: a multilayer perceptron, a deep neural network (DNN), a convolutional neural network (CNN), a recurrent neural network (RNN), a long short-term memory (LSTM). For example, the artificial neural network can comprise at most 30, at most 15, or at most 5 trainable intermediate layers. Such a network architecture is particularly computing-efficient and nonetheless enables a sufficiently accurate estimation.

According to one embodiment, the artificial neural network may be implemented as an adaptive neuro-fuzzy inference system (ANFIS). Such an inference system may be based on a Takagi-Sugeno controller and/or Tsukamoto controller and/or may comprise an array of fuzzy if-then rules, which can be trained to approximate nonlinear functions. The architecture of the ANFIS may comprise, for example, five layers, among them a so-called fuzzification layer as a first of the five layers. This embodiment enables the advantages of an artificial neural network to be unified with the advantages of a fuzzy logic in a single framework.

According to one embodiment, the measurement data may comprise at least first measurement data and second measurement data. In this case, the first measurement data may indicate a volume-dependent course of a concentration of a first respiratory gas in the respiratory airflow depending on the respiratory air volume and the second measurement data may indicate a volume-dependent course of a concentration of a second respiratory gas, differing from the first respiratory gas, in the respiratory airflow depending on the respiratory air volume. Accordingly, the output data may indicate a concentration of the first respiratory gas and the second respiratory gas in the blood of the patient. This embodiment enables the simultaneous estimation of the concentrations of various respiratory gases in the blood of the patient, for example, of carbon dioxide and oxygen.

According to one embodiment, the input data may comprise first input data generated from the first measurement data and second input data generated from the second measurement data. The first input data may be input here into a first artificial neural network and first output data, which indicate the concentration of the first respiratory gas in the blood of the patient, may be output by the first artificial neural network. Analogously thereto, the second input data may be input into a second artificial neural network and second output data, which indicate the concentration of the second respiratory gas in the blood of the patient, may be output by the second artificial neural network. Accordingly, the output data may comprise the first output data and the second output data. The weights of the machine learning model may be, for example, weights of the first or second artificial neural network. The two artificial neural networks may differ from one another or correspond to one another in their architecture and/or in their weights and/or may have been trained separately from one another. For example, at least one or each of the two artificial neural networks may correspond in its architecture with the above-described network and/or can be implemented as an adaptive neuro-fuzzy inference system. Alternatively, the first and the second input data may be input into a common artificial neural network, for example into the above-described network.

For example, the first input data may comprise a matrix of first values for various first parameters with respect to the volume-dependent course of the first measurement data and/or the second input data may comprise a matrix of second values for various second parameters with respect to the volume-dependent course of the second measurement data. The first parameters may correspond in their number and/or their type with the second parameters and/or may deviate from the second parameters. It is possible that at least one of the first values is determined using a first mathematical function, which approximately defines at least one section of the volume-dependent course of the first measurement data (for example, from various time steps), and/or at least one of the second values is determined using a second mathematical function, which approximately defines at least one section of the volume-dependent course of the second measurement data (for example, from various time steps).

### BRIEF DESCRIPTION OF THE DRAWINGS

Embodiments of the invention are described hereinafter with reference to the appended drawings. Neither the description nor the drawings are to be understood as a restriction of the scope of the invention. In the drawings:

FIG. 1 shows a ventilator according to one embodiment of the invention.

FIG. 2 shows a flow chart to illustrate an embodiment of a method for estimating a concentration of a respiratory gas in the blood of a patient.

FIG. 3 shows a flow chart to illustrate an embodiment of a method for training a machine learning module for a ventilator.

FIG. 4 shows a capnogram for use in a method according to one embodiment of the invention.

FIG. 5 shows a machine learning module for use in a method according to one embodiment of the invention.

FIG. 6 shows a flow chart to illustrate the generation of training data sets in a method according to one embodiment of the invention.

The figures are solely schematic and are not to scale. If identical reference signs are used in different drawings, these reference signs designate identical or identically-acting features.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

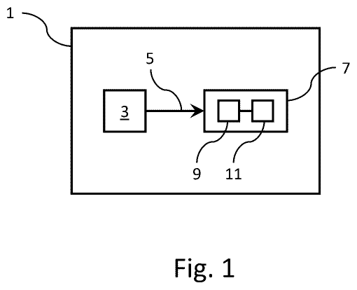

FIG. 1 shows a ventilator 1 for the invasive and/or noninvasive ventilation of a patient. The ventilator 1 comprises a sensor system 3 for generating measurement data 5, and a data processing device 7 having a memory 9 and a processor 11, which is configured to execute a computer program stored in the memory 9 for processing the measurement data 5 in at least one of the methods described below.

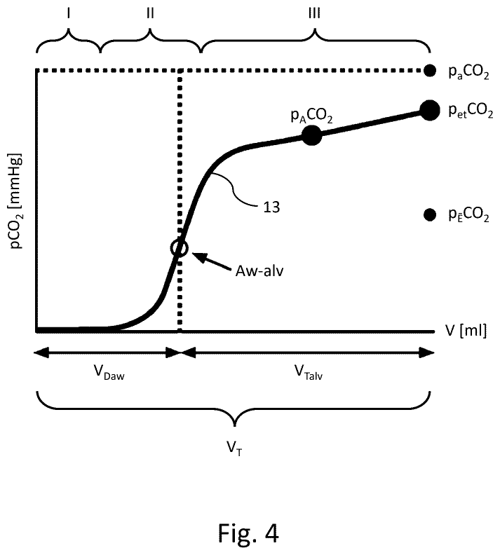

The measurement data 5 define a volume-dependent course 13 (see FIG. 4) of a concentration of a respiratory gas (for example, pCO₂) in a respiratory airflow exhaled by the patient depending on a respiratory air volume exhaled by the patient.

In particular, the measurement data 5 can indicate the volume-dependent course 13 with respect to a single breath of the patient. Moreover, it is possible that the measurement data 5 indicate a positive end-expiratory pressure (abbreviated PEEP), which is associated with the volume-dependent course 13, for the ventilation of the patient.

For example, the measurement data 5—as shown in FIG. 4—can code a capnogram produced noninvasively by volumetric capnography, which indicates the volume-dependent course 13 of the concentration of carbon dioxide as the respiratory gas. Additionally or alternatively, the measurement data 5 can code an oxigram produced noninvasively by volumetric oxigraphy, which indicates the volume-dependent course 13 of the concentration of oxygen as the respiratory gas.

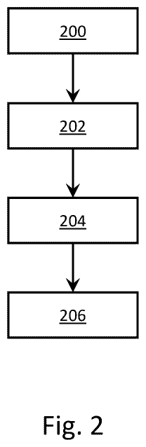

FIG. 2 shows an example of a method for estimating the concentration of the respiratory gas in the blood of the patient. The method is carried out by the data processing device 7 and comprises the following steps.

In a step 200, the measurement data 5 are received.

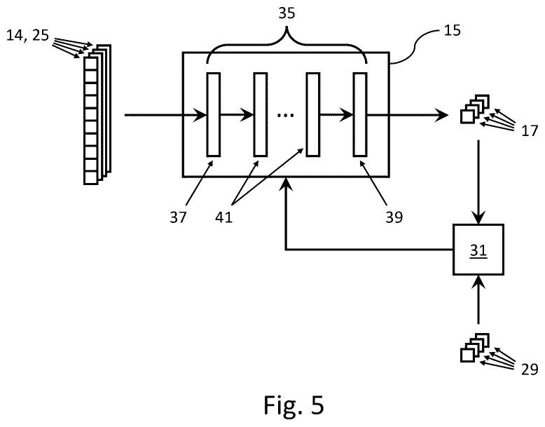

In a step 202, input data 14 are produced from the measurement data 5 (see also FIG. 5). The input data 14 comprise a matrix of values for various parameters with respect to the volume-dependent course.

In a step 204, the input data 14 are input into a machine learning module 15, which was trained to convert the input data 14 into output data 17, which indicate the (estimated) concentration of the respiratory gas in the blood of the patient (“module” can be understood above and below as a software and/or hardware module). For example, the output data 17 can comprise an estimated value for the arterial carbon dioxide partial pressure (pₐCO₂) and/or an estimated value for the arterial oxygen partial pressure (pₐO₂).

In a step 206, the output data 17 are output by the machine learning module 15.

Subsequently, the output data 17 can additionally be further processed, for example, to display the output data 17 in a suitable manner on a display of the ventilator 1 and/or to transmit them via a wireless and/or wired data communication connection to an external device, e.g., a server, a PC, a laptop, a tablet, a smart phone, or a smart watch.

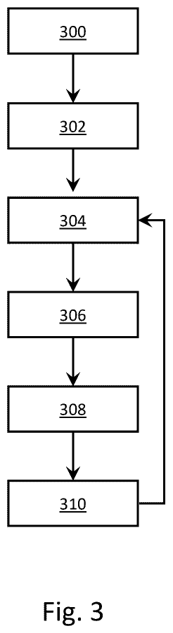

FIG. 3 shows an example of a method for training the machine learning module 15. It is possible that the method is likewise carried out by the data processing device 7. Additionally or alternatively, the method can be carried out by another data processing device outside the ventilator 1. The method comprises the following steps.

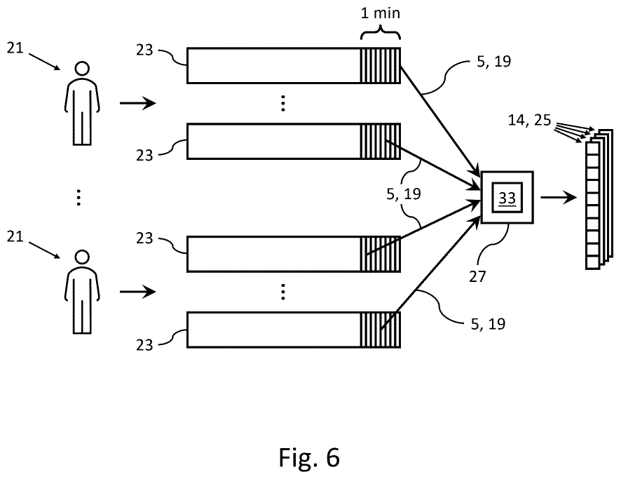

In a step 300, multiple measurement data sets 19, which each comprise the measurement data 5, are received (see also FIG. 6). The measurement data 5 of the various measurement data sets 19 are at least partially associated with different patients 21. For example, the measurement data sets 19 can have been generated during the machine ventilation of the patient 21 in multiple controlled ventilation steps 23 of a specific length of, for example, 2 minutes, 5 minutes, or 10 minutes. In this case, each measurement data set 19 can comprise the measurement data 5 for precisely one breath of the respective patient 21.

In a step 302, multiple training data sets 25 are generated from the measurement data sets 19 of the various patients 21, wherein each training data set 25 is associated with one of the patients 21 and comprises a matrix of values for various parameters with respect to the respective volume-dependent course 13. The training data sets 25 can be generated, for example, by a suitably configured selection module 27, which can be implemented as a further machine learning module. It is possible that only measurement data sets 19 which were each generated in a specific time interval of the ventilation steps 23, for example, in the last minute, as shown in FIG. 6, are used to generate the training data sets 25. With a controlled respiration duration of two seconds, 30 measurement data sets therefore result per ventilation step 23 of each patient 21. A training data set 25 can then be generated, for example, from each of these measurement data sets 19.

In a step 304, each training data set 25 is input as input data 14 into the machine learning module 15. The machine learning module 15 converts the input data 14 into output data 17, which indicate the (estimated) concentration of the respiratory gas in the blood of the respective patient 21.

In a step 306, the output data 17 are output by the machine learning module 15.

In a step 308, a deviation of the output data 17 from target data 29, which are associated with the respective training data set 25 or the respective input data 14, is determined.

Finally, in a step 310, the weights of the machine learning module 15 are adapted in an optimization method to reduce the deviation, for example, in a gradient method with back propagation.

Steps 308, 310 can be carried out, for example, by a suitably configured optimization module 31.

The machine learning module 15 is expediently trained in multiple successive time steps until the deviation reaches an acceptable value. Steps 304 to 310 can be carried out in each time step here, for example.

As shown in FIG. 5 and FIG. 6, the matrix can be a one-dimensional vector and/or can comprise values for at least 2, at least 5, or at least 10 different parameters, in particular for 11 or 12 different parameters. The number of the parameters is not to be excessively large, for example, not greater than 15, 20, or 30, in order to keep the consumption of computing resources low. The number of the values in the matrix can correspond to the number of the various parameters, i.e. each value in the matrix can define precisely one of the various parameters.

Depending on the embodiment, the various parameters can comprise at least one of the following parameters with respect to the respective patient (see also FIG. 4): a total gas volume of the respiratory gas exhaled by the patient during a single breath; a total volume of respiratory air comprising the respiratory gas exhaled by the patient during a single breath, also called breath volume or tidal volume (V_{T}); a respiratory minute volume; an alveolar ventilation; an airway dead space (V_{Daw}); a mixed expiratory partial pressure of the respiratory gas (for example, [symbol] ); an end-tidal partial pressure of the respiratory gas (for example, pₑₜCO₂); a positive end-expiratory pressure for ventilating the patient.

Moreover, the volume-dependent course 13 of the respective measurement data 5 can be divided into at least three successive characteristic ventilation phases during a single breath of the respective patient. In this case, the various parameters can comprise at least one of the following parameters: a first respiratory gas volume as a volume of the respiratory gas exhaled by the patient in a first exhalation phase I; a second respiratory gas volume as a volume of the respiratory gas exhaled by the patient in a second exhalation phase II; a third respiratory gas volume as a volume of the respiratory gas exhaled by the patient in a third exhalation phase III; an average slope of the volume-dependent course 13 in at least one of the exhalation phases I, II, III, in particular in the third exhalation phase III.

Optionally, the three respiratory gas volumes can each be normalized by division by the breath volume. In this case, the various parameters can comprise the respective normalized respiratory gas volume.

The values for these parameters can be at least partially calculated from the measurement data 5 using a special mathematical function 33. The mathematical function 33 can also be used to determine the three exhalation phases I, II, III. Alternatively, the various parameters can be at least partially determined using an additional machine learning module.

The mathematical function 33 can be an approximation of at least one section of the volume-dependent course 13. The approximation can be determined, for example, by a regression analysis of measurement data 5, which were generated in multiple successive time steps, for example, during multiple breaths, with respect to a patient. A particularly accurate and computing-efficient approximation can be achieved if the mathematical function 33 is determined according to the Levenberg-Marquardt algorithm.

The machine learning module 15 can be implemented, for example, as an artificial neural network 35 (see FIG. 5) having an input layer 37 for inputting the input data 14, an output layer 39 for outputting the output data 17, and at least one trainable intermediate layer 41 for converting the input data 14 into the output data 17. Alternatively, the machine learning module 15 can be implemented as a combination of multiple artificial neural networks. Other machine learning algorithms are also possible, such as a decision tree, a random forest, a k-nearest neighbor algorithm, a support vector machine, a Bayes classifier, a k-means algorithm, a genetic algorithm, a kernel regression algorithm, or a discriminant analysis algorithm.

To enable rapid training—for example, on the ventilator 1—and the use of cost-effective hardware, the network 35 is not to be excessively complex. An architecture having at most 10 trainable intermediate layers 41 proved in experiments to be a good compromise between efficiency and accuracy. A network 35 in the form of an adaptive neuro-fuzzy inference system (ANFIS) is particularly favorable. However, significantly more complex DNN architectures are also possible.

The machine learning module 15 can also be trained to estimate the concentration of various respiratory gases in the blood of the patient at the same time.

In this case, the measurement data 5 can comprise, for example, first measurement data and second measurement data, wherein the first measurement data indicate a volume-dependent course 13 of a concentration of a first respiratory gas, for example, carbon dioxide, in the respiratory airflow depending on the respiratory air volume and the second measurement data indicate a volume-dependent course of a concentration of a second respiratory gas, for example oxygen, in the respiratory airflow depending on the respiratory air volume. Accordingly, the training data sets 25 or the input data 14 can be generated from the first and the second measurement data and the output data 17 can indicate an (estimated) concentration of the first respiratory gas and the second respiratory gas in the blood of the respective patient.

For this purpose, the input data 14 can be input either into a common artificial neural network 35 or into two separately trainable or trained artificial neural networks. In the second case, for example, first input data for a first network can be generated from the first measurement data and second input data for a second network can be generated from the second measurement data. Accordingly, first output data, which indicate the concentration of the first respiratory gas in the blood of the patient, are output by the first network and second output data, which indicate the concentration of the second respiratory gas in the blood of the patient, are output by the second network.

The training of the machine learning module 15 can comprise, for example, the following steps.

Initially, blood samples are collected from various patients and a collection time is noted for each blood sample collection.

The blood samples are analyzed to determine an actual arterial carbon dioxide and/or oxygen partial pressure of the respective patient.

Simultaneously with and/or shortly before and/or shortly after the blood sample collection, the carbon dioxide or oxygen partial pressure in the respiratory airflow exhaled by the respective patient is sensorially detected during multiple breaths at least during the exhalation. Moreover, the flow of the respiratory airflow is sensorially detected at least during the exhalation.

The respiratory air volume exhaled by the patient is determined for each breath by integration of the flow.

On this basis, corresponding volumetric capnograms or oxigrams are generated from the time-based courses of the carbon dioxide or oxygen partial pressure in the respiratory airflow and the respiratory air volume.

Subsequently, in an optimization method, for example with the aid of a regression analysis, a mathematical function is determined which approximately defines the curves in the capnograms or oxigrams.

Various parameters, which describe specific features of the curves, are then determined on the basis of the mathematical function.

These parameters are used together with the respective values of the actual arterial carbon dioxide or oxygen partial pressure to train the machine learning module 15.

It was possible to confirm the practicality of the method, inter alia, in the following experiment.

In 14 lung-flushed experimental animals, the arterial carbon dioxide partial pressure (PₐCO₂) was continuously detected by means of an optical intravascular catheter. At the same time, a capnogram was detected during each breath. The animals were mechanically ventilated using fixed settings, wherein the positive end-expiratory pressure was varied in multiple steps from 0 to 22 cmH₂O. The resulting 8599 data points—in each case a pₐCO₂ value paired with a set of 12 parameters which were derived from the capnogram of a breath—were input into an ANFIS-Modell. The data points were divided into a first set of 7370 data points (85%) for training the model and a second set of 1229 data points (15%) for testing the trained model. The ANFIS analysis was repeated in 10 independent steps, wherein the data points to be input were each selected according to the random principle.

The Bland-Altman diagram for the 10 ANFIS models tested independently of one another resulted in a mean deviation of 0.03±0.03 mmHg between estimated and actual pₐCO₂ value with a correspondence range of 2.25±0.42 mmHg and a mean square deviation of 1.15±0.06 mmHg. The estimation was therefore sufficiently accurate. A concordance index of 95.5% (four-quadrant diagram) or 94.3% (polar diagram) was calculated, which indicates a good trend capacity.

The advantages of the method can be attributed to the following points, among other things.

Firstly, volume-based measurement data instead of time-based measurement data are used. In particular with the aid of capnography or oxigraphy, numerous informative parameters with respect to the gas exchange can be monitored. These parameters represent a more robust database for the assessment of the gas exchange than solely time-based measurement data with respect to the exhaled respiratory gas flow.

Furthermore, it was possible to show that the parallel evaluation of multiple parameters results in a more accurate estimation than the evaluation of a single parameter (for example, of the end-tidal carbon dioxide partial pressure).

The use of a machine learning module moreover offers the advantage that nonlinear hidden information in conjunction with the pulmonary gas exchange during each breath can also be taken into consideration.

Finally, it is to be noted that terms such as “has”, “comprises”, “includes”, “having”, etc. do not exclude other elements or steps and indefinite articles such as “a” or “an” do not exclude multiples.

Furthermore, it is to be noted that features or steps which are described with reference to one of the preceding embodiments can also be used in combination with features or steps which are described with reference to others of the preceding embodiments.

Reference signs in the claims are not to be understood as a restriction of the scope of the subject matter defined by the claims.

### SOURCES

1. Guggenberger H, Lenz G, Federle R (1989): Early detection of inadvertent oesophageal intubation: pulse oximetry vs. capnography. Acta Anaesthesiol Scand 33:112-115.
2. Walsh BK, Crotwell DN, Restrepo RD (2011): Capnography/Capnometry during mechanical ventilation: 2011. Respir Care 56:503-509.
3. Rackley CR (2020): Monitoring during mechanical ventilation. Respir Care 65:832-846.
4. McDonald MJ, Montgomery VL, Cerrito PB, Parrish CJ, Boland KA, Sullivan JE (2002): Comparison of end-tidal CO2 and PₐCO2 in children receiving mechanical ventilation. Pedi Crit Care Med 3:244-249.
5. McSwain SD, Hamel DS, Smith PB, Gentile MA, Srinivasan S, Meliones JN, Cheifetz IM (2010): End-tidal and arterial carbon dioxide measurements correlate across all levels of physiologic dead space. Respir Care 55:288-293.
6. Razi E, Moosavi GA, Omidi K, Saebi AK, Razi A (2012): Correlation of end-tidal carbon dioxide with arterial carbon dioxide in mechanically ventilated patients. Arch Trauma Research 1:58.
7. Satoh K, Ohashi A, Kumagai M, Sato M, Kuji A, Joh S (2015): Evaluation of differences between PₐCO2 and ETCO2 by age as measured during general anesthesia with patients in a supine position. J Anesth doi.org/10.1155/2015/710537.
8. Tavernier B, Rey D, Thevenin D, Triboulet JP, Scherpereel P (1997): Can prolonged expiration manoeuvres improve the prediction of arterial PCO2 from end-tidal PCO2? British J Anaesth 78:536-540.
9. Plewa MC, Sikora S, Engoren M, Tome D, Thomas J, Deuster A (1995): Evaluation of capnography in nonintubated emergency department patients with respiratory distress. Acad Emerg Med 2:901-908.
10. Trevisanuto D, Giuliotto S, Cavallin F, Doglioni N, Toniazzo S, Zanardo V (2012): End-tidal carbon dioxide monitoring in very low birth weight infants: correlation and agreement with arterial carbon dioxide. Ped Pulmonol 47:367-372.
11. Nassar BS, Schmidt GA (2017): Estimating arterial partial pressure of carbon dioxide in ventilated patients: how valid are surrogate measures? Annals Am Thorac Soc 14:1005-1014.
12. Bhardwaj A, Kishore S, Pandey DK (2022): Artificial Intelligence in Biological Sciences. Life 12:1430.
13. Briganti G, Le Moine O (2020): Artificial intelligence in medicine: today and tomorrow. Front in Med 7:27.
14. Miller DD, Brown EW (2018): Artificial intelligence in medical practice: the question to the answer? Am J Med 131:129-133.
15. Johri AM, Mantella LE, Jamthikar AD, Saba L, Laird JR, Suri JS (2021): Role of artificial intelligence in cardiovascular risk prediction and outcomes: comparison of machine-learning and conventional statistical approaches for the analysis of carotid ultrasound features and intra-plaque neovascularization. Int J Cardiovasc Imag 37: 3145-3156.
16. Suarez-Sipmann F, Bohm SH, Tusman G (2014): Volumetric capnography: the time has come. Curr Opin Crit Care 20:333-339.
17. Tusman G, Suarez-Sipmann F, Bohm SH, Borges JB, Hedenstierna G (2011): Capnography reflects ventilation/perfusion distribution in a model of acute lung injury. Acta Anaesthesiol Scand 55:597-606
18. Suárez-Sipmann F, Villar J, Ferrando C, Sánchez-Giralt JA, Tusman G (2021): Monitoring Expired CO2 Kinetics to Individualize Lung-Protective Ventilation in Patients With the Acute Respiratory Distress Syndrome. Front Physiol https://doi.org/10.3389/fphys.2021.785014.
19. Tusman G, Acosta CM, Wallin M, Hallbäck M, Esperatti M, Peralta G et al (2022): Perioperative continuous noninvasive cardiac output monitoring in cardiac surgery patients by a novel capnodynamic method. J Cardiothorac Vasc Anesth 36:2900-2907.
20. Suarez-Sipmann F, Böhm SH, Tusman G, Pesch T, Thamm O, Reissmann H, Hedenstierna G (2007): Use of dynamic compliance for open lung positive end-expiratory pressure titration in an experimental study. Crit Care Med 35:214-22.
21. Tusman G, Bohm SH, Suarez-Sipmann F, Scandurra A, Hedenstierna G (2010): Lung recruitment and positive end-expiratory pressure have different effects on CO2 elimination in healthy and sick lungs. Anesth Analg 111:968-977.
22. Tusman G, Scandurra A, Böhm SH, Suarez-Sipmann F, Clara F (2009): Model fitting of volumetric capnograms improves calculations of airway dead space and slope of phase III. J Clin Monit Comput 23:197-206.
23. Palmer TEA, Cumpston PHV, Foster WJ, Jones RDM (1995): Continuous intravascular blood gas analysis during aortic aneurysm repair: the Paratrend 7®. Anaesth Intensive Care 23:200-202.
24. Venkatesh B, Clutton-Brock TH, Hendry SP (1995): Evaluation of the Paratrend 7 intravascular blood gas monitor during cardiac surgery: comparison with the C4000 in-line blood gas monitor during cardiopulmonary bypass. J Cardiothorac Vasc Anesth 9:412-419.
25. J. Jang, “ANFIS: adaptive-network-based fuzzy inference systems,” IEEE Trans. Syst. Man. Cybern., vol. 23, pp. 665-85, 1993.
26. T. Takagi and M. Sugeno, “Fuzzy identification of systems and its applications to modeling and control.,” IEEE Trans. Syst. Man. Cybern., vol. 15, pp. 116-32, 1985.
27 M. Sugeno and G. T. Kang, “Structure identification of fuzzy model.,” Fuzzy Sets and Systems, vol. 28, pp. 15-33, 1988.
28 J. Jang and N. Gulley, Fuzzy Logic Toolbox: User's Guide. Natick, MA: The Math Works, Inc, 2000.
29. Critchley LA, Lee A, Ho AMH (2010): A critical review of the ability of continuous cardiac output monitors to measure trends in cardiac output. Anesth Analg 111:1180-1192.
30. Critchley LA, Yang XX, Lee A (2011): Assessment of trending ability of cardiac output monitors by polar plot methodology. J Cardiothorac Vasc Anesth 25:536-546.
31. Montenij LJ, Buhre WF, De Jong SA, Harms JH, Van Herwaarden JA, Kruitwagen CL, De Waal EE: Arterial pressure waveform analysis versus thermodilution cardiac output measurement during open abdominal aortic aneurysm repair: A prospective observational study. Eur J Anaesthesiol| 2015; 32:13-19.
32. Belenkiy SM, Baker WL, Batchinsky AI, Mittal S, Watkins T, Salinas J, Cancio LC: Multivariate analysis of the volumetric capnograph for PₐCO2 estimation. Int J Burns & Trauma 2015; 5:66.
33. Khemani RG, Celikkaya EB, Shelton CR, Kale D, Ross PA, Wetzel RC, Newth CJ (2014): Algorithms to estimate PₐCO2 and pH using noninvasive parameters for children with hypoxemic respiratory failure. Respir Care 59:1248-1257.
34. Engoren M, Plewa M, O'Hara D, Kline JA: Evaluation of capnography using a genetic algorithm to predict PₐCO2. Chest 2005; 127:579-584.

### LIST OF REFERENCE NUMERALS

1 ventilator
3 sensor system
5 measurement data
7 data processing device
9 memory III third exhalation phase
11 processor
13 volume-dependent course
14 input data
15 machine learning module
17 output data
19 measurement data set
21 patient
23 ventilation step
25 training data set
27 selection module
29 target data
31 optimization module
33 mathematical function
35 artificial neural network
37 input layer
39 output layer
41 intermediate layer
200 receiving measurement data
202 generating input data
204 inputting input data
206 outputting output data
300 receiving measurement data sets
302 generating training data sets
304 inputting input data
306 outputting output data
308 determining a deviation
310 adapting weights
Aw-alv inflection point (boundary between airways and alveoli)
I first exhalation phase
II second exhalation phase
pCO₂ carbon dioxide partial pressure
PₐCO₂ arterial carbon dioxide partial pressure
P_{A}CO₂ alveolar carbon dioxide partial pressure
PₑₜCO₂ end-tidal carbon dioxide partial pressure
[symbol] mixed expiratory carbon dioxide partial pressure
V respiratory air volume
V_{Daw} airway dead space
V_{T} breath volume
V_{Talv} alveolar tidal volume

What is claimed is:

1. A computer-implemented method for estimating a concentration (pₐCO₂) of a respiratory gas in the blood of a patient, wherein the method comprises:
receiving measurement data, which indicate a volume-dependent course of a concentration (pCO₂) of the respiratory gas in a respiratory airflow exhaled by the patient depending on a respiratory air volume (V) exhaled by the patient;
generating input data from the measurement data, wherein the input data comprise a matrix of values for various parameters with respect to the volume-dependent course;
inputting the input data into a machine-learning module, which was trained to convert the input data into output data, which output data indicate a concentration (pₐCO₂) of the respiratory gas in the blood of the patient;
outputting the output data by way of the machine learning module.

1. A computer-implemented method for training a machine learning module for a medical device, wherein the method comprises:
receiving multiple measurement data sets, which each comprise measurement data that indicate a volume-dependent course of a concentration (pCO₂) of a respiratory gas in a respiratory airflow exhaled by a patient depending on a respiratory air volume (V) exhaled by the patient, wherein the measurement data of various measurement data sets are at least partially associated with different patients;
generating multiple training data sets from the measurement data sets, wherein each training data set is associated with one of the patients and comprises a matrix of values for various parameters with respect to the volume-dependent course;
inputting each training data set as input data into the machine learning module, which is configured to convert the input data into output data, which output data indicate a concentration (pₐCO₂) of the respiratory gas in the blood of the respective patient;
outputting the output data by way of the machine learning module;
determining a deviation of the output data from target data, which are associated with the respective training data set;
adapting weights of the machine learning module in an optimization method to reduce the deviation.

2. The method of claim 1,
wherein the measurement data indicate the volume-dependent course with respect to a single breath of the patient; and/or
wherein the measurement data furthermore indicate a positive end-expiratory pressure, associated with the volume-dependent course, for ventilating the patient.

3. The method of claim 1,
wherein a mathematical function, which approximately defines at least one section of the volume-dependent course, is determined using the measurement data, wherein at least one of the values is calculated in a matrix using the mathematical function.

4. The method of claim 4,
wherein the measurement data were generated in multiple successive time steps and the mathematical function is determined using the measurement data from various time steps; and/or
wherein the mathematical function is determined according to the Levenberg-Marquardt algorithm.

5. The method of claim 1,
wherein the various parameters comprise at least one of the following parameters: a total volume of the respiratory gas exhaled during a single breath by the patient; a total volume (V_{T}) of respiratory air comprising the respiratory gas exhaled during a single breath by the patient; a respiratory minute volume; an alveolar ventilation; an airway dead space (V_{Daw}); a mixed expiratory partial pressure ( [symbol] ) of the respiratory gas; an end-tidal partial pressure (pₑₜCO₂) of the respiratory gas; a positive end-expiratory pressure for ventilating the patient; and/or
wherein the volume-dependent course is divided into at least three successive characteristic exhalation phases (I, II, III) during a single breath of the patient, wherein the various parameters comprise at least one of the following parameters: a first respiratory gas volume as a volume of the respiratory gas exhaled by the patient in a first (I) of the exhalation phases (I, II, III); a second respiratory gas volume as a volume of the respiratory gas exhaled by the patient in a second (II) of the exhalation phases (I, II, III); a third respiratory gas volume as a volume of the respiratory gas exhaled by the patient in a third (III) of the exhalation phases (I, II, III); a slope of the volume-dependent course in at least one of the exhalation phases (I, II, III).

6. The method of claim 6,
wherein at least one normalized respiratory gas volume is determined by dividing one of the three respiratory gas volumes by a total gas volume (VT), exhaled by the patient during the single breath, of respiratory air comprising the respiratory gas, wherein the parameters comprise the at least one normalized respiratory gas volume.

7. The method of claim 1, wherein the machine learning module comprises an artificial neural network.

8. The method of claim 8, wherein the artificial neural network is implemented as an adaptive neuro-fuzzy inference system.

9. The method of claim 1,
wherein the measurement data comprise at least first measurement data and second measurement data, wherein the first measurement data indicate a volume-dependent course of a concentration (pCO₂) of a first respiratory gas in the respiratory airflow depending on the respiratory air volume (V) and the second measurement data indicate a volume-dependent course of a concentration of a second respiratory gas in the respiratory airflow depending on the respiratory air volume (V);
wherein the output data indicate a concentration (pₐCO₂) of the first respiratory gas and a concentration of the second respiratory gas in the blood of the patient.

10. The method of claim 10,
wherein the input data comprise first input data generated from the first measurement data and second input data generated from the second measurement data;
wherein the first input data are input into a first artificial neural network and first output data, which indicate the concentration (pₐCO₂) of the first respiratory gas in the blood of the patient, are output by the first artificial neural network;
wherein the second input data are input into a second artificial neural network and second output data, which indicate the concentration of the second respiratory gas in the blood of the patient, are output by the second artificial neural network;
wherein the output data comprise the first output data and the second output data.

11. A data processing device, wherein the device comprises a processor which is configured to carry out the method of claim 1.

12. A medical device, wherein the device comprises:
a sensor system for generating measurement data, which indicate a volume-dependent course of a concentration (pCO₂) of a respiratory gas in a respiratory airflow exhaled by a patient depending on a respiratory air volume (V) exhaled by the patient;
the data processing device of claim 12.

13. A computer program, wherein the program comprises commands which prompt a processor, upon execution of the computer program by the processor, to carry out the method of claim 1.

14. A computer-readable medium, on which the computer program of claim 14 is stored.

15. The method of claim 2,
wherein the measurement data indicate the volume-dependent course with respect to a single breath of the patient; and/or
wherein the measurement data furthermore indicate a positive end-expiratory pressure, associated with the volume-dependent course, for ventilating the patient.

16. The method of claim 2,
wherein a mathematical function, which approximately defines at least one section of the volume-dependent course, is determined using the measurement data, wherein at least one of the values is calculated in a matrix using the mathematical function.

17. The method of claim 17,
wherein the measurement data were generated in multiple successive time steps and the mathematical function is determined using the measurement data from various time steps; and/or
wherein the mathematical function is determined according to the Levenberg-Marquardt algorithm.

18. The method of claim 2,
wherein the various parameters comprise at least one of the following parameters: a total volume of the respiratory gas exhaled during a single breath by the patient; a total volume (V_{T}) of respiratory air comprising the respiratory gas exhaled during a single breath by the patient; a respiratory minute volume; an alveolar ventilation; an airway dead space (V_{Daw}); a mixed expiratory partial pressure ( [symbol] ) of the respiratory gas; an end-tidal partial pressure (pₑₜCO₂) of the respiratory gas; a positive end-expiratory pressure for ventilating the patient; and/or
wherein the volume-dependent course is divided into at least three successive characteristic exhalation phases (I, II, III) during a single breath of the patient, wherein the various parameters comprise at least one of the following parameters: a first respiratory gas volume as a volume of the respiratory gas exhaled by the patient in a first (I) of the exhalation phases (I, II, III); a second respiratory gas volume as a volume of the respiratory gas exhaled by the patient in a second (II) of the exhalation phases (I, II, III); a third respiratory gas volume as a volume of the respiratory gas exhaled by the patient in a third (III) of the exhalation phases (I, II, III); a slope of the volume-dependent course in at least one of the exhalation phases (I, II, III).

19. The method of claim 19,
wherein at least one normalized respiratory gas volume is determined by dividing one of the three respiratory gas volumes by a total gas volume (VT), exhaled by the patient during the single breath, of respiratory air comprising the respiratory gas, wherein the parameters comprise the at least one normalized respiratory gas volume.
